(19) United States
(12) Patent Application Publication
(10) Pub. No.: US 2024/0316145 A1
(43) Pub. Date: Sep. 26, 2024

(54) SECRETOGLOBIN FAMILY 1D MEMBER 2 (SCGB1D2) PROTEIN INHIBITS GROWTH OF BORRELIA BURGDORFERI AND AFFECTS SUSCEPTIBILITY TO LYME DISEASE

(71) Applicants: The Board of Trustees of the Leland Stanford Junior University, Stanford, CA (US); University of Helsinki, Helsinki (FI)

(72) Inventors: Michal C. Tal, Cupertino, CA (US); Grace Blacker, Stanford, CA (US); Sarah Galloway, Stanford, CA (US); Paige Hansen, Palo Alto, CA (US); Hanna Maria Ollila, Palo Alto, CA (US); Satu Strausz, Stanford, CA (US); Tuomas Aivelo, Stanford, CA (US); Nasa Sinnott-Armstrong, Redwood City, CA (US)

(21) Appl. No.: 18/125,007

(22) Filed: Mar. 22, 2023

(51) Int. Cl. A61K 38/17; A61P 31/04; C12Q 1/18

(52) U.S. Cl. CPC A61K 38/1703; A61P 31/04; C12Q 1/18

(57) ABSTRACT

Compositions and methods are provided for inhibiting the growth of Borrelia species. Inhibitors for use in the method comprise human secretoglobin family 1D member 2 (SCGB1D2) protein, and variants and mimetics thereof.

### BACKGROUND

Lyme disease, i.e. borreliosis, is an infectious disease caused by bacteria of the genus Borrelia, and transmitted by ticks. While most individuals have subsiding or short term infection others develop severe infection which requires intensive antibiotic treatment and may result in chronic illness. In Europe, three distinct genospecies cause Lyme disease: Borrelia afzelii which is linked to skin complication, B. garinii, linked to neurological sequelae, and rarely B. burgdorferi linked to arthritic symptoms, whereas B. burgdorferi is the only genospecies in Northern America. Seasonality of the disease is well characterized and an increasing number of patients for Lyme disease have emerged during the past decades. Yet the biological risk factors and disease mechanisms for infection or for severe illness are still only partially understood.

A genome-wide association study (GWAS) on spirochetal diseases including Lyme disease assessed by ICD10 and 9 codes, explored the phenotypic and genetic risk factors with the goal to fine map the most significant genetic associations and to understand underlying biology.

### SUMMARY

Compositions and methods are provided for inhibiting the growth of Borrelia species. Inhibitors for use in the method comprise human secretoglobin family 1D member 2 (SCGB1D2) protein, and variants and mimetics thereof, which are referred to herein as SCGB1D2 agents. It is shown herein that SCGB1D2 agents inhibit the growth of Borrelia species, including, without limitation, Borrelia burgdorferi, and other Borrelia sp. causative of Lyme disease, e.g. Borrelia afzelli, B. garinii, etc.

In some embodiments, an SCGB1D2 agent is a polypeptide. In some embodiments the polypeptide comprises a human reference sequence SCGB1D2 protein or biologically active fragment thereof. In some embodiments, a polypeptide SCGB1D2 agent competes for binding to a human reference sequence SCGB1D2 protein. In some embodiments an SCGB1D2 agent is a polynucleotide encoding an SCGB1D2 protein. In some embodiments, an SCGB1D2 agent is obtained by screening candidate agents for activity in mimicking the activity of a human SCGB1D2 protein.

In some embodiments, an effective dose of an SCGB1D2 agent is administered to an individual for the prevention and/or treatment of Borrelia infection. The individual may be homozygous for the SCGB1D2 reference allele, or is optionally an individual homozygous or heterozygous for a variant allele of SCGB1D2, for example an allele comprising a substitution of proline at position 53 to leucine (P53L). In some embodiments, administration of an SCGB1D2 agent is topical, e.g. a cream, gel, lotion, etc. In some embodiments, administration is systemic. An effective concentration of an SCGB1D2 agent may be from about 0.1 mg/ml up to about 1000 mg/ml, from about 0.5 mg/ml to about 500 mg/ml, from about 1 mg/ml to about 250 mg/ml, e.g. from about 0.1 mg/ml, from about 0.5 mg/ml, from about 1 mg/ml, from about 5 mg/ml, from about 10 mg/ml, from about 20 mg/ml, from about 50 mg/ml, up to about 1000 mg/ml, up to about 500 mg/ml. For example, a topical formulation comprising an effective concentration of an SCGB1D2 agent may be administered prior to or shortly after a suspected exposure to Borrelia, at sites of tick bites, and the like.

In some embodiments a therapeutic formulation is provided comprising an SCGB1D2 agent in a pharmaceutically acceptable excipient. The therapeutic formulation may be provided in a unit dose, comprising an effective dose of the SCGB1D2 agent. In some embodiments the formulation is a topical formulation.

In some embodiments, methods are provided for screening a candidate agent for efficacy in inhibition of growth of Borrelia species, the methods comprising designing an agent to mimic SCGB1D2 activity, and determining its effectiveness in inhibition of growth. In some such embodiments, a candidate agent is a mutagenized SCGB1D2 protein or peptide derived therefrom, e.g. fragments, truncated version of the protein, variants comprising random or designed amino acid substitutions, and the like.

The examples provided herein demonstrate that human reference sequence SCGB1D2 protein inhibits the growth of Borrelia burgdorferi, whereas a variant allele SCGB1D2 with deleterious missense mutation is less effective at inhibition of growth. SCGB1D2 is a novel host defense factor present in the skin and secretions, which protects against Borrelia infection and spirochetal diseases including Lyme disease. This finding provides a therapeutic avenue for drug development to prevent and treat Lyme disease. Although humans in general can benefit from the methods disclosed herein, individuals with variant SCGB1D2 that have reduced natural protection from Borrelia infection may be selected for treatment.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1C. FIG. 1a) Manhattan plot for the genome-wide association study (GWAS) of Lyme disease (LD) including 5,248 LD cases and 337,251 controls. For each genetic variant, the x-axis shows chromosomal position, while y-axis shows the −log 10(P)-value. The horizontal line indicates the genome-wide significance threshold of P=5.0×10⁻⁸. Two genetic loci were identified at a genome-wide significance level: SCGB1D2 and HLA-DQB1. FIG. 1b) Locus Zoom plot shows associated P-values on the −log₁₀ scale on the vertical axis, and the chromosomal position along the horizontal axis. Purple diamond indicates single nucleotide polymorphism (SNP) at a locus with the strongest associated evidence. Linkage disequilibrium (LD, r² values) between the lead SNP and the other SNPs are indicated by color. FIG. 1c) Schematic illustration for the protein structure of SCGB1D2 where a missense variant rs2232950 is causing an amino acid substitution from proline (Pro) to leucine (Leu). The structure is alpha-helical and forms an antiparallel dimer of the two monomers. There is a cavity which can accommodate small to medium sized ligands like steroids and phospholipids between the two dimers.

FIG. 2. Volcano plot of Phenome-wide associations (PheWAS) from rs2232950 and 2,202 disease endpoints from FinnGen. Each point represents a trait. Vertical axis presents associated P-values at −log 10 scale and the horizontal axis shows beta values. Other spirochetal diseases contain individuals who have been treated at hospital inpatient or outpatient clinics.

FIGS. 3A-3J. Borrelia burgdorferi (Bb) was incubated alone (black), or with either reference (Ref) or variant (Var) SCGB1D2 recombinant protein at 2 μg/mL, 4 μg/mL, 8 μg/mL or 16 μg/mL. IncuCyte analysis showing the count of green fluorescent protein (GFP)-expressing Bb per image with reference SCGB1D2 protein at 24 h time point FIG. 3a) and at 72 h time point FIG. 3b). Count of GFP-expressing Bb spirochetes per image over time with all concentrations of reference SCGB1D2 protein FIG. 3c). Count of GFP-expressing Bb spirochetes per image over time with SCGB1D2 reference and SCGB1D2 P53L recombinant proteins at 8 μg/ml and 16 μg/mL concentration FIG. 3d). Comparison of reference SCBG1D2 and variant SCGB1D2 P53L at 8 μg/mL and 16 μg/mL concentrations over time, and FIG. 3e) at 72 hours. Representative IncuCyte images for each treatment condition at 140 hours, scale bars at 200 μm (FIG. 3f-j). ***P=0.001, **P<0.01, *P<0.05 and ns, not significant.

FIG. 4. Phenome-wide association (PheWAS) from publicly available data from the OpenTargets platform are visualized by trait and their −log 10(P-values) in the y-axis. Colors represent trait categories.

FIGS. 5A-5B. FIG. 5a) Manhattan plot for the genome-wide association study (GWAS) for syphilis including 719 individuals with syphilis diagnosis and 341,780 controls. HLA-DRB5 association seen also with syphilis is different from our HLA association in Lyme disease, and the P-value from the lead variant from Lyme disease HLA lead variant rs9273375 is P=0.45. FIG. 5b) Regional association of SCGB1D2 locus in syphilis. The variant with lowest P-value is marked here with a diamond. P-value for Lyme disease variant rs2232950 is not significant (P=0.6).

FIG. 6. Expression pattern of SCGB1D2 across human tissues. We obtained RNA expression data from the GTEx project and examined the expression profile of SCGB1D2 across tissues. A total of 701 individuals with expression values showed the highest expression in the skin (sun exposed) and skin (not sun exposed).

FIG. 7. Single cell sequencing data shows SCGB1D2 expression is specific for sweat gland cells. Data from He et al.

FIG. 8. Timescale analysis of Borrelia burgdorferi growth inhibition by SCGB1D2 P53L over 72 h hours. Y-axis represents green fluorescent protein (GFP) count per frame and X-axis represents time. Concentrations tested are 2 to 16 μg/mL.

FIGS. 9A-9B. Borrelia burgdorferi (Bb) spirochetes expressing green fluorescent protein (GFP) were incubated with FIG. 9a) with or without propidium iodide (PI). FIG. 9b) either 8 μg/mL or 16 μg/mL of reference (Ref) or variant (Var) SCGB1D2 protein in the presence of PI to measure Bb death by SCGB1D2. After 24 hours of incubation, an aliquot of each culture was analyzed by flow cytometry. Overall one-way ANOVA was used comparing Bb with PI and Bb with PI and SCGB1D2 proteins (ANOVA F(4, 14)=3.185, P=0.0467, Dunnett's multiple comparisons test P=0.082). ****P<0.0001; ns, not significant.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Five of the named Borrelia species are regularly found in human patients. These causative agents of human Lyme disease are B. burgdorferi sensu stricto in North America and Europe, B. garinii, B. bavariensis, B. afzelii in Europe and Asia, and B. spielmanii in Europe. Typing systems such as those provided herein that accurately characterize species and strains within species are crucial for epidemiological, clinical, and evolutionary studies.

Borrelia burgdorferi sensu lato is a group of spirochetes belonging to the genus Borrelia in the family of Spirochaetaceae. The spirochete is transmitted between reservoir hosts by ticks of the family Ixodidae. Infection with B. burgdorferi in humans may cause Lyme disease, or Lyme borreliosis, which is the most common vector-borne disease in North America and Europe. More than 40 species have been described in the genus Borrelia. These include 20 Borrelia species within the B. burgdorferi sensu lato complex and more than 20 Borrelia species associated with relapsing fever. The genus Borrelia possesses certain genetic and phenotypic characteristics that are unique among prokaryotes. Borrelia cells are helical with dimensions of 0.2 to 0.5 μm by 10 to 30 μm, allowing them to be easily distinguished from other eubacteria based on the phenotypic features common for all spirochetes. Borrelia can also be differentiated from other pathogenic spirochetes such as treponemes and leptospires on the basis of morphological traits, including the wavelength of the cell coils, the presence or absence of terminal hooks, the shape of the cell poles, and the number of periplasmic flagella. However, it is almost impossible to phenotypically distinguish different species within the Borrelia genus. Therefore, the identification and differentiation of different Borrelia species and strains is largely dependent on analyses of their genetic characteristics or on serology.

Lyme Disease. Lyme disease is a tick-transmitted infection caused by Borrelia burgdorferi. Early symptoms include an erythema migrans rash, which may be followed weeks to months later by neurologic, cardiac, or joint abnormalities. Diagnosis is primarily clinical in early-stage disease, but serologic testing by the methods described herein can help diagnose cardiac, neurologic, and rheumatologic complications that occur later in the disease. Treatment is with antibiotics such as doxycycline or ceftriaxone, and may involve additional agents in later stages of the disease.

Lyme disease is transmitted primarily by 4 Ixodes sp worldwide: Ixodes scapularis (the deer tick) in the northeastern and north central US, I. pacificus in the western US, I. ricinus in Europe., I. persulcatus in Asia. B. burgdorferi enters the skin at the site of the tick bite. After 3 to 32 days, the organisms migrate locally in the skin around the bite, spread via the lymphatics to cause regional adenopathy or disseminate in blood to organs or other skin sites. Initially, an inflammatory reaction (erythema migrans) occurs before significant antibody response to infection (serologic conversion).

Lyme disease has 3 stages: Early localized, early disseminated, and late. The early and late stages are usually separated by an asymptomatic interval. Erythema migrans (EM), the hallmark and best clinical indicator of Lyme disease, is the first sign of the disease. It occurs in at least 75% of patients, beginning as a red macule or papule at the site of the tick bite, usually on the proximal portion of an extremity or the trunk (especially the thigh, buttock, or axilla), between 3 and 32 days after a tick bite. The area expands, often with clearing between the center and periphery resembling a bull's eye, to a diameter ˜50 cm. Darkening erythema may develop in the center, which may be hot to the touch and indurated. Without therapy, EM typically fades within 3 to 4 wk.

Symptoms of early-disseminated disease begin days or weeks after the appearance of the primary lesion, when the bacteria spread through the body. Soon after onset, nearly half of untreated patients develop multiple, usually smaller annular secondary skin lesions without indurated centers. Cultures of biopsy samples of these secondary lesions have been positive, indicating dissemination of infection. Patients also develop a musculoskeletal, flu-like syndrome, consisting of malaise, fatigue, chills, fever, headache, stiff neck, myalgias, and arthralgias that may last for weeks. Because symptoms are often nonspecific, the diagnosis is frequently missed if EM is absent. Symptoms are characteristically intermittent and changing, but malaise and fatigue may linger for weeks. Some patients develop symptoms of fibromyalgia. Resolved skin lesions may reappear faintly, sometimes before recurrent attacks of arthritis, in late-stage disease.

Neurologic abnormalities develop in about 15% of patients within weeks to months of EM (generally before arthritis occurs), commonly last for months, and usually resolve completely. Most common are lymphocytic meningitis or meningoencephalitis, cranial neuritis, and sensory or motor radiculoneuropathies, alone or in combination. Myocardial abnormalities occur in about 8% of patients within weeks of EM. They include fluctuating degrees of atrioventricular block (1st-degree, Wenckebach, or 3rd-degree) and, rarely, myopericarditis with chest pain, reduced ejection fractions, and cardiomegaly.

In untreated Lyme disease, the late stage begins months to years after initial infection. Arthritis develops in about 60% of patients within several months, occasionally up to 2 yr, of disease onset (as defined by EM). Intermittent swelling and pain in a few large joints, especially the knees, typically recur for several years. Affected knees commonly are much more swollen than painful; they are often hot, but rarely red. Baker cysts may form and rupture. Malaise, fatigue, and low-grade fever may precede or accompany arthritis attacks. In about 10% of patients, knee involvement is chronic. Other late findings (occurring years after onset) include an antibiotic-sensitive skin lesion (acrodermatitis chronica atrophicans) and chronic CNS abnormalities, either polyneuropathy or a subtle encephalopathy with mood, memory, and sleep disorders.

Conventional treatment alternatives may vary with stage of disease but typically include amoxicillin, doxycycline, and ceftriaxone. In late-stage disease, antibiotics eradicate the bacteria, relieving the arthritis in most people. However, individuals may have persistent arthritis even after the infection has been eliminated because of continued inflammation and may be further treated with anti-inflammatory agents.

Individuals in which Lyme disease is diagnosed may be administered an SCGB1D2 agent, alone or in combination with antibiotics, anti-IgE therapy, mast cell stabilizer, or an antihistamine such as, but not limited to, cimetidine, ranitidine, Benadryl, diphenhydramine, loratadine, doxepin, thioperamide, and clobenpropit.

Secretoglobin family 1D member 2 is a member of the lipophilin subfamily, part of the uteroglobin superfamily, and is an ortholog of prostatein, the major secretory glycoprotein of the rat ventral prostate gland. Lipophilin gene products are widely expressed in normal tissues, especially in endocrine-responsive organs. Assuming that human lipophilins are the functional counterparts of prostatein, they may be transcriptionally regulated by steroid hormones, with the ability to bind androgens, other steroids and possibly bind and concentrate estramustine, a chemotherapeutic agent widely used for prostate cancer. Although the gene has been reported to be on chromosome 10, this sequence appears to be from a cluster of genes on chromosome 11 that includes mammaglobin 2. The human protein refseq is NP_006542 in Genbank, and the mRNA refseq is NM_006551.

The wild type (reference) human sequence is (SEQ ID NO:1) MKLSVCLLLV TLALCCYQAN AEFCPALVSE LLDFFFISEP LFKLSLAKFD APPEAVAAKL GVKRCTDQMS LQKRSLIAEV LVKILKKCSV

A variant allele with amino acid substitution P53L has the sequence (SEQ ID NO:2) MKLSVCLLLVTLALCCYQANAEFCPALVSELLDFFFISEPLFKLSLAKFDAPLEAVAAKLGVKRCT DQMSLQKRSLIAEVLVKILKKCSV.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “sequence identity,” as used herein in reference to polypeptide or DNA sequences, refers to the subunit sequence identity between two molecules. When a subunit position in both of the molecules is occupied by the same monomeric subunit (e.g., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent.

By “parent polypeptide”, “parent protein”, “precursor polypeptide”, or “precursor protein” as used herein is meant an unmodified polypeptide that is subsequently modified to generate a variant. A parent polypeptide may be a wild-type (or native) polypeptide, or a variant or engineered version of a wild-type polypeptide. Parent polypeptide may refer to the polypeptide itself, compositions that comprise the parent polypeptide, or the amino acid sequence that encodes it.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. “Amino acid analogs” refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acid modifications disclosed herein may include amino acid substitutions, deletions and insertions, particularly amino acid substitutions. Variant proteins may also include conservative modifications and substitutions at other positions of the cytokine and/or receptor (e.g., positions other than those involved in the affinity engineering). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: Ala, Pro, Gly, Gln, Asn, Ser, Thr; Group II: Cys, Ser, Tyr, Thr; Group III: Val, lie, Leu, Met, Ala, Phe; Group IV: Lys, Arg, His; Group V: Phe, Tyr, Trp, His; and Group VI: Asp, Glu. Further, amino acid substitutions with a designated amino acid may be replaced with a conservative change.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

In some embodiments, a polypeptide is conjugated to additional molecules to provide desired pharmacological properties such as extended half-life. In one embodiment, a polypeptide can be fused to the Fc domain of IgG, albumin, or other molecules to extend its half-life, e.g. by pegylation, glycosylation, and the like as known in the art. In some embodiments the polypeptide is conjugated to a polyethylene glycol molecules or “PEGylated.” The molecular weight of the PEG conjugated to the polypeptide ligand include but are not limited to PEGs having molecular weights between 5 kDa and 80 kDa, in some embodiments the PEG has a molecular weight of approximately 5 kDa, in some embodiments the PEG has a molecular weight of approximately 10 kDa, in some embodiments the PEG has a molecular weight of approximately 20 kDa, in some embodiments the PEG has a molecular weight of approximately 30 kDa, in some embodiments the PEG has a molecular weight of approximately 40 kDa, in some embodiments the PEG has a molecular weight of approximately 50 kDa, in some embodiments the PEG has a molecular weight of approximately 60 kDa in some embodiments the PEG has a molecular weight of approximately 80 kDa. In some embodiments, the molecular mass is from about 5 kDa to about 80 kDa, from about 5 kDa to about 60 kDa, from about 5 kDa to about 40 kDa, from about 5 kDa to about 20 kDa. The PEG conjugated to the polypeptide sequence may be linear or branched. The PEG may be attached directly to the polypeptide, or attached via a linker molecule. The processes and chemical reactions necessary to achieve PEGylation of biological compounds is well known in the art.

The polypeptide can be acetylated at the N-terminus, using methods known in the art, e.g. by enzymatic reaction with N-terminal acetyltransferase and, for example, acetyl CoA. The polypeptide can be acetylated at one or more lysine residues, e.g. by enzymatic reaction with a lysine acetyltransferase. See, for example Choudhary et al. (2009). Science. 325 (5942): 834-840.

Fc-fusion can also endow alternative Fc receptor mediated properties in vivo. The “Fc region” can be a naturally occurring or synthetic polypeptide that is homologous to an IgG C-terminal domain produced by digestion of IgG with papain. IgG Fc has a molecular weight of approximately 50 kDa. The ortholog IL-2 polypeptides can include the entire Fc region, or a smaller portion that retains the ability to extend the circulating half-life of a chimeric polypeptide of which it is a part. In addition, full-length or fragmented Fc regions can be variants of the wild-type molecule. That is, they can contain mutations that may or may not affect the function of the polypeptides; as described further below, native activity is not necessary or desired in all cases.

In other embodiments, a polypeptide can comprise a sequence that functions as an antigenic tag, such as a FLAG sequence. FLAG sequences are recognized by biotinylated, highly specific, anti-FLAG antibodies, as described herein (see also Blanar et al., Science 256: 1014, 1992; LeClair et al., Proc. Natl. Acad. Sci. USA 89:8145, 1992). In some embodiments, the chimeric polypeptide further comprises a C-terminal c-myc epitope tag.

As described above, the proteins of the invention may exist as a part of a chimeric polypeptide. In addition to, or in place of, the heterologous polypeptides described above, a nucleic acid molecule of the invention can contain sequences encoding a “marker” or “reporter.” Examples of marker or reporter genes include β-lactamase, chloramphenicol acetyltransferase (CAT), adenosine deaminase (ADA), aminoglycoside phosphotransferase (neo1, G418r), dihydrofolate reductase (DHFR), hygromycin-B-hosphotransferase (HPH), thymidine kinase (TK), lacz (encoding β-galactosidase), and xanthine guanine phosphoribosyltransferase (XGPRT). As with many of the standard procedures associated with the practice of the invention, skilled artisans will be aware of additional useful reagents, for example, of additional sequences that can serve the function of a marker or reporter.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's diseased cell, e.g., a sample comprising polynucleotides and/or polypeptides that is obtained from a patient's diseased cell (e.g., a cell lysate or other cell extract comprising polynucleotides and/or polypeptides); and a sample comprising diseased cells from a patient. A biological sample comprising a diseased cell from a patient can also include non-diseased cells.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. The term “therapeutic effect” refers to the reduction, elimination, or prevention of the disease, symptoms of the disease, or side effects of the disease in the subject.

As used herein, a “therapeutically effective amount” refers to that amount of the therapeutic agent sufficient to prevent, treat or manage a disease or disorder. A therapeutically effective amount may refer to the amount of therapeutic agent sufficient to delay or minimize the onset of disease. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the terms “prevent”, “preventing” and “prevention” refer to the prevention of the recurrence or onset of one or more symptoms of a disorder in a subject as result of the administration of a prophylactic or therapeutic agent.

As used herein, the term “in combination” refers to the use of more than one prophylactic and/or therapeutic agents. When administered in combination, each component can be administered at the same time or sequentially in any order at different points in time. Thus, each component can be administered separately but sufficiently closely in time so as to provide the desired therapeutic effect. The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Dosage and frequency may vary depending on the half-life of the agent in the patient. It will be understood by one of skill in the art that such guidelines will be adjusted for the molecular weight of the active agent, the clearance from the blood, the mode of administration, and other pharmacokinetic parameters. The dosage may also be varied for localized administration, e.g. topical, intranasal, inhalation, etc., or for systemic administration, e.g. i.m., i.p., i.v., oral, and the like.

An active agent can be administered by any suitable means, including topical, oral, parenteral, intrapulmonary, and intranasal. Parenteral infusions include intramuscular, intravenous (bolus or slow drip), intraarterial, intraperitoneal, intrathecal or subcutaneous administration. An agent can be administered in any manner which is medically acceptable. This may include injections, by parenteral routes such as topical, intradermal, intravenous, intravascular, intraarterial, subcutaneous, intramuscular, intratumor, intraperitoneal, intraventricular, intraepidural, or others as well as oral, nasal, ophthalmic, rectal, or topical. Sustained release administration is also specifically included in the disclosure, by such means as depot injections or erodible implants.

As noted above, an agent can be formulated with an a pharmaceutically acceptable carrier (one or more organic or inorganic ingredients, natural or synthetic, with which a subject agent is combined to facilitate its application). A suitable carrier includes sterile saline although other aqueous and non-aqueous isotonic sterile solutions and sterile suspensions known to be pharmaceutically acceptable are known to those of ordinary skill in the art. An “effective amount” refers to that amount which is capable of preventing infection or further bacterial growth. An effective amount can be determined on an individual basis and will be based, in part, on consideration of the symptoms to be treated and results sought. An effective amount can be determined by one of ordinary skill in the art employing such factors and using no more than routine experimentation.

An agent can be administered as a pharmaceutical composition comprising a pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds can also be made by methods known to one of ordinary skill in the art. As used herein, “methods known to one of ordinary skill in the art” may be identified through various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., www.acs.org may be contacted for more details). Chemicals that are known but not commercially available in catalogs may be prepared by custom chemical synthesis houses, where many of the standard chemical supply houses (e.g., those listed above) provide custom synthesis services.

The active agents of the invention and/or the compounds administered therewith are incorporated into a variety of formulations for therapeutic administration. In one aspect, the agents are formulated into pharmaceutical compositions by combination with appropriate, pharmaceutically acceptable carriers or diluents, and are formulated into preparations in solid, semi-solid, liquid or gaseous forms, such as tablets, capsules, powders, granules, ointments, solutions, suppositories, injections, inhalants, gels, microspheres, and aerosols. As such, administration of the active agents and/or other compounds can be achieved in various ways, usually by oral administration. The active agents and/or other compounds may be systemic after administration or may be localized by virtue of the formulation, or by the use of an implant that acts to retain the active dose at the site of implantation.

In pharmaceutical dosage forms, the active agents and/or other compounds may be administered in the form of their pharmaceutically acceptable salts, or they may also be used alone or in appropriate association, as well as in combination with other pharmaceutically active compounds. The agents may be combined, as previously described, to provide a cocktail of activities. The following methods and excipients are exemplary and are not to be construed as limiting the invention.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Formulations are typically provided in a unit dosage form, where the term “unit dosage form,” refers to physically discrete units suitable as unitary dosages for human subjects, each unit containing a predetermined quantity of active agent in an amount calculated sufficient to produce the desired effect in association with a pharmaceutically acceptable diluent, carrier or vehicle. The specifications for the unit dosage forms of the present invention depend on the particular complex employed and the effect to be achieved, and the pharmacodynamics associated with each complex in the host.

The pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Depending on the patient and condition being treated and on the administration route, the active agent may be administered in dosages of 0.01 mg to 500 mg/kg body weight per day, e.g. about 0.5 mg, about 1 mg, about 5 mg, about 10 mg, about 20 mg/day or more for an average person. Dosages will be appropriately adjusted for pediatric formulation.

In some embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. The nature of the carrier can be either soluble or insoluble for purposes of the invention.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEENTM, PLURONICSTM or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Topical formulations include an acceptable vehicle to act as a diluent, dispersant or carrier for the active agents, so as to facilitate distribution and uptake when the composition is applied to the skin. Vehicles other than or in addition to water can include liquid or solid emollients, solvents, humectants, thickeners and powders. The vehicle will usually form 5% to 99.9%, preferably from 25% to 80%, about 40% to 60%, by weight of the composition, and can, in the absence of other adjuncts, form the balance of the composition.

The compositions of the invention may also contain additives and adjuvants which are conventional in the topical, pharmaceutical or dermatological field, such as hydrophilic or lipophilic gelling agents, hydrophilic or lipophilic active agents, preservatives, antioxidants, solvents, fragrances, fillers, bactericides, odor absorbers and dyestuffs or colorants. The amounts of these various additives and adjuvants are those conventionally used in the field, and, for example, range from 0.01% to 10% of the total weight of the composition. Depending on their nature, these additives and adjuvants may be introduced into the fatty phase or into the aqueous phase.

Exemplary oils which may be used according to this invention include mineral oils (liquid petrolatum), plant oils (liquid fraction of karite butter, sunflower oil), animal oils (perhydrosqualen(e), synthetic oils (purcellin oil), silicone oils (cyclomethicone) and fluoro oils (perfluoropolyethers). Fatty alcohols, fatty acids (stearic acid) and waxes (paraffin wax, carnauba wax and beeswax) may also be used as fats.

Emulsifiers which may be used include glyceryl stearate, polysorbate 60, PEG-6/PEG-32/glycol stearate mixture, etc. Solvents which may be used include the lower alcohols, in particular ethanol and isopropanol, and propylene glycol. Hydrophilic gelling agents include carboxyvinyl polymers (carbomer), acrylic copolymers such as acrylate/alkylacrylate copolymers, polyacrylamides, polysaccharides, such as hydroxypropylcellulose, natural gums and clays, and, as lipophilic gelling agents, representative are the modified clays such as bentones, fatty acid metal salts such as aluminum stearates and hydrophobic silica, or ethylcellulose and polyethylene.

An oil or oily material may be present, together with an emollient to provide either a water-in-oil emulsion or an oil-in-water emulsion, depending largely on the average hydrophilic-lipophilic balance (HLB) of the emollient employed. Levels of such emollients may range from about 0.5% to about 50%, preferably between about 5% and 30% by weight of the total composition. Emollients may be classified under such general chemical categories as esters, fatty acids and alcohols, polyols and hydrocarbons.

Esters may be mono- or di-esters. Acceptable examples of fatty di-esters include dibutyl adipate, diethyl sebacate, diisopropyl dimerate, and dioctyl succinate. Acceptable branched chain fatty esters include 2-ethyl-hexyl myristate, isopropyl stearate and isostearyl palmitate. Acceptable tribasic acid esters include triisopropyl trilinoleate and trilauryl citrate. Acceptable straight chain fatty esters include lauryl palmitate, myristyl lactate, oleyl eurcate and stearyl oleate. Preferred esters include coco-caprylate/caprate (a blend of coco-caprylate and coco-caprate), propylene glycol myristyl ether acetate, diisopropyl adipate and cetyl octanoate.

Suitable fatty alcohols and acids include those compounds having from 10 to 20 carbon atoms. Especially preferred are such compounds such as cetyl, myristyl, palmitic and stearyl alcohols and acids.

Among the polyols which may serve as emollients are linear and branched chain alkyl polyhydroxyl compounds. For example, propylene glycol, sorbitol and glycerin are preferred. Also useful may be polymeric polyols such as polypropylene glycol and polyethylene glycol. Butylene and propylene glycol are also especially preferred as penetration enhancers.

Exemplary hydrocarbons which may serve as emollients are those having hydrocarbon chains anywhere from 12 to 30 carbon atoms. Specific examples include mineral oil, petroleum jelly, squalene and isoparaffins.

Another category of functional ingredients within the topical compositions of the present disclosure are thickeners. A thickener will usually be present in amounts anywhere from 0.1 to 20% by weight, preferably from about 0.5% to 10% by weight of the composition. Exemplary thickeners are cross-linked polyacrylate materials available under the trademark Carbopol. Gums may be employed such as xanthan, carrageenan, gelatin, karaya, pectin and locust beans gum. Under certain circumstances the thickening function may be accomplished by a material also serving as a silicone or emollient. For instance, silicone gums in excess of 10 centistokes and esters such as glycerol stearate have dual functionality.

Other adjunct minor components may also be incorporated into the topical compositions. These ingredients may include coloring agents, opacifiers and perfumes. Amounts of these other adjunct minor components may range anywhere from 0.001% up to 20% by weight of the composition. A typical composition of the present disclosure is formulated as a cream, which may be applied topically once or twice daily.

In use, a quantity of the composition, for example from 1 to 100 ml, is applied to a site of interest from a suitable container or applicator and, if necessary, it is then spread over and/or rubbed into the site using the hand or fingers or a suitable device. The product may be specifically formulated for use as a treatment for a specific area, e.g. a site of a tick bite, etc.

The topical composition of the present disclosure can be formulated in any form suitable for application to the site of interest, including a lotion, cream, gel, or the like. The composition can be packaged in any suitable container to suit its viscosity and intended use by the consumer. For example, a lotion or cream can be packaged in a bottle, or a propellant-driven aerosol device or a container fitted with a pump suitable for finger and/or manual operation. When the composition is a cream, it can simply be stored in a non-deformable bottle or squeeze container, such as a lidded jar, a pump dispenser, or a tube. The present disclosure accordingly also provides a closed container containing a composition as herein defined.

Toxicity of the active agents can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in further optimizing and/or defining a therapeutic dosage range and/or a sub-therapeutic dosage range (e.g., for use in humans). The exact formulation, route of administration and dosage can be chosen by the individual physician in view of the patient's condition.

Expression construct: The coding sequences of an SCGB1D2 protein may be introduced on an expression vector into a cell for expression. For example, a coding sequence may be introduced into a target cell using CRISPR technology. CRISPR/Cas9 system can be directly applied to human cells by transfection with a plasmid that encodes Cas9 and sgRNA. The viral delivery of CRISPR components has been extensively demonstrated using lentiviral and retroviral vectors. Gene editing with CRISPR encoded by non-integrating virus, such as adenovirus and adenovirus-associated virus (AAV), has also been reported. Recent discoveries of smaller Cas proteins have enabled and enhanced the combination of this technology with vectors that have gained increasing success for their safety profile and efficiency, such as AAV vectors.

The nucleic acid encoding SCGB1D2 can be inserted into a vector for expression and/or integration. Many such vectors are available. The vector components generally include, but are not limited to, one or more of the following: an origin of replication, one or more marker genes, an enhancer element, a promoter, and a transcription termination sequence. Vectors include viral vectors, plasmid vectors, integrating vectors, and the like.

Expression vectors may contain a selection gene, also termed a selectable marker. This gene encodes a protein necessary for the survival or growth of transformed host cells grown in a selective culture medium or a truncated gene encoding a surface marker that allows for antibody based detection. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, or (d) enable surface antibody based detection for isolation via fluoresences activating cell sorting (FACS) or magnetic separation.

Nucleic acids are “operably linked” when placed into a functional relationship with another nucleic acid sequence. For example, DNA for a signal sequence is operably linked to DNA for a polypeptide if it is expressed as a preprotein that signals the secretion of the polypeptide; a promoter or enhancer is operably linked to a coding sequence if it affects the transcription of the sequence; and a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

Expression vectors will contain a promoter that is recognized by the host organism and is operably linked to the SCGB1D2 construct coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus LTR (such as murine stem cell virus), hepatitis-B virus and Simian Virus 40 (SV40), from heterologous mammalian promoters, e.g., the actin promoter, PGK (phosphoglycerate kinase), or an immunoglobulin promoter, or from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes may be increased by inserting an enhancer sequence into the vector. Enhancers are cis-acting elements of DNA, usually about from 10 to 300 bp in length, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, α-fetoprotein, and insulin). Typically, however, one will use an enhancer from a eukaryotic virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence, but is preferably located at a site 5′ from the promoter.

Expression vectors for use in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

Suitable host cells for cloning a construct are the prokaryotic, yeast, or other eukaryotic cells described above. Examples of useful mammalian host cell lines are mouse L cells (L-M[K-], ATCC #CRL-2648), monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); Chinese hamster ovary cells/-DHFR (CHO); mouse Sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2).

Host cells can be transfected with the above-described expression vectors for construct expression. Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression, and will be apparent to the ordinarily

### Methods of Treatment

Methods are provided for treating a Borrelia infection, including Lyme disease resulting from a Borrelia infection, in a subject in need thereof, which methods comprise administering a therapeutically effective dose of an SCGB1D2 agent to the subject. Administration may be topical. Administration may be parenteral. The individual may have been diagnosed with a Borrelia infection prior to treatment. Alternatively the individual may be at risk of a Borrelia infection, e.g. following a tick bite, an individual frequently outdoors, living in a tick infested area, etc. Treatment may be combined with a suitable antibiotic, e.g. amoxicillin, cefotaxime, ceftriaxone, cefuroxime, doxycycline, erythromycin, azithromycin, penicillin and tetracycline. The treatment can be to cure, heal, alleviate, relieve, alter, remedy, ameliorate, palliate, improve or affect Lyme disease or symptoms associated with Lyme disease.

In some embodiments the subject is homozygous or heterozygous for a variant allele of SCGB1D2, for example an allele comprising a substitution of proline at position 53 to leucine (P53L). The subject may be genotyped by conventional methods, e.g. SNP analysis, sequencing, and the like as known in the art.

For some subjects treated at an early stage of active Borrelia infection, treatment for a normal period (e.g., a week) can cure the infection completely. Some patients, particularly those diagnosed with later stages of Lyme disease, may have persistent or recurrent symptoms. For such subjects, extended therapy (e.g., one, two, three, four, or more weeks of treatment) can be administered.

The response to therapy may be monitored, including subjective (e.g., self-report of symptoms) and objective measurements of Lyme disease symptoms. For example, the methods provided herein can be used in parallel with clinical observations of, or a subject's self-reporting of, tick bite, erythema migrans (or bull-eye shaped rash), skin lesion, pain, fever, headache, swelling, or other symptoms associated with Lyme disease. In some embodiments, these methods can be used in parallel with Western blot analysis or serological assays for the presence of Borrelia-specific antibodies.

Combination therapies utilizing an SCGB1D2 agent and one or more additional therapeutic agents may show synergistic effects, e.g., a greater therapeutic effect than would be observed using either an SCGB1D2 agent or one or more additional therapeutic agents alone as a monotherapy.

According to aspects, combination therapies include: (1) pharmaceutical compositions that include an SCGB1D2 agent in combination with one or more additional therapeutic agents; and (2) co-administration of an SCGB1D2 agent with one or more additional therapeutic agents wherein the SCGB1D2 agent and the one or more additional therapeutic agents have not been formulated in the same composition. When using separate formulations, an SCGB1D2 agent may be administered at the same time, intermittent times, staggered times, prior to, subsequent to, or combinations thereof, with reference to the administration of the one or more additional therapeutic agents.

Combination treatments can allow for reduced effective dosage and increased therapeutic index of the SCGB1D2 agent and the one or more additional therapeutic agents.

In particular aspects, compositions of the present invention are formulated for topical application, for example to be administered at the site of a tick bite. A topical formulation can be an ointment, lotion, cream or gel in particular aspects. Topical dosage forms such as ointment, lotion, cream or gel bases are described in Remington: The Science and Practice of Pharmacy, 21st Ed., Lippincott Williams & Wilkins, 2006, p. 880-882 and p. 886-888; and in Allen, L. V. et al., Ansel's Pharmaceutical Dosage Forms and Drug Delivery Systems, 8th Ed., Lippincott Williams & Wilkins, 2005, p. 277-297.

Antibiotics may be administered in combination with an SCGB1D2 agent, e.g. antibiotics with the classes of aminoglycosides; carbapenems; and the like; penicillins, e.g. penicillin G, penicillin V, methicillin, oxacillin, carbenicillin, nafcillin, ampicillin, etc. penicillins in combination with β-lactamase inhibitors, cephalosporins, e.g. cefaclor, cefazolin, cefuroxime, moxalactam, etc: tetracyclines; cephalosporins; quinolones; lincomycins; macrolides; sulfonamides; glycopeptides including the anti-infective antibiotics vancomycin, teicoplanin, telavancin, ramoplanin and decaplanin. Derivatives of vancomycin include, for example, oritavancin and dalbavancin (both lipoglycopeptides). Telavancin is a semi-synthetic lipoglycopeptide derivative of vancomycin (approved by FDA in 2009). Other vancomycin analogs are disclosed, for example, in WO 2015022335 A1 and Chen et al. (2003) PNAS 100(10): 5658-5663, each herein specifically incorporated by reference. Non-limiting examples of antibiotics include vancomycin, linezolid, azithromycin, daptomycin, colistin, eperezolid, fusidic acid, rifampicin, tetracyclin, fidaxomicin, clindamycin, lincomycin, rifalazil, and clarithromycin.

Mast cell stabilizing drugs inhibit the release of allergic mediators from mast cells and are used clinically, e.g. to prevent allergic reactions. Mast cells have a role in allergic diseases because of hypersensitive response to substances that induces an allergic reaction, for example the release of preformed chemical mediators such as histamine, synthesis of lipid mediators such as PGs and LTs, production of cytokines and chemokines, etc. Mast cell stabilizers may be used at conventional dosages to reduce undesirable mast cell activation may be administered in combination with an SCGB1D2 agent.

The most commonly used mast cell stabilizer is disodium cromoglycate, which inhibits IgE-dependent mast cell activation. Natural product mast stabilizers include, for example, Luteolin; Diosmetin; Quercetin; Fisetin; Kaempferol; Ginkgetin; Silymarin; Scopletin; Scaporone; Artekeiskeanol; Selinidin; Cinnamic acid; Ellagic acid; Magnolol and honokiol; Resveratrol; Polydatin; Curcumin; Mangostin-α, -β and -γ; Parthenolide; Sesquiterpene lactones; Monoterpenes; Sinomenine; Indoline; Xestospongin; Theanine; etc. Biologic inhibitors also include, for example, complement-derived peptide C3a, and the C3a9 peptide derived therefrom. Other anti-allergic peptides have been identified, e.g. LVA, LSY, RVS, ETI, TDG, RVV and GFW, which inhibited antigen-stimulated release of β-hexosaminidase from RBL-2H3 cells.

Synthetic and semi-synthetic mast cell stabilizers are also known in the art. For example indanone sesquiterpenes have been modified, and include the indanone, pterosin Z. Synthetic stabilizers include, for example, Compound 13, R112, ER-27317, U63A05, WHI-131, Hypothemycin, Midostaurin (PKC412), CP99994, K1, Ro 20-1724, rolipram and Siguazodan, Fullerenes, Vacuolin-1, CMT-3, OR-1384, OR-1958, TLCK, TPCK, Bromoenol lactone (BEL), Cerivastatin, atorvastatin and fluvastatin, Nilotinib, etc.

The term antihistamine is given its normal usage, i.e. a class of drug that opposes the activity of histamine receptors in the body, which are subclassified according to the histamine receptor that they act upon. The two largest classes of antihistamines are H1-antihistamines and H2-antihistamines. H1-antihistamines work by binding to histamine H1 receptors in mast cells, smooth muscle, and endothelium in the body as well as in the tuberomammillary nucleus in the brain. H2-antihistamines bind to histamine H2 receptors in the upper gastrointestinal tract, primarily in the stomach.

The majority of H1-antihistamines are receptor antagonists. Clinically, H1-antihistamines are used to treat allergic reactions and mast cell-related disorders and may be administered in combination with an SCGB1D2 agent. Examples of H1 antagonists include: Acrivastine, Azelastine, Bilastine, Bromodiphenhydramine, Brompheniramine, Buclizine, Carbinoxamine, Cetirizine, Chlorodiphenhydramine, Chlorpheniramine, Clemastine, Cyclizine, Cyproheptadine, Desloratadine (Aerius), Dexbrompheniramine, Dexchlorpheniramine, Dimenhydrinate, Dimetindene, Diphenhydramine, Doxylamine, Ebastine, Embramine, Fexofenadine, Hydroxyzine, Levocabastine, Levocetirizine, Loratadine, Meclizine, Mirtazapine, Olopatadine, Orphenadrine, Phenindamine, Pheniramine, Phenyltoloxamine, Promethazine Quetiapine, Rupatadine, Tripelennamine, Triprolidine. Inverse H1 agonists include, for examples, Levocetirizine, Desloratadine, Pyrilamine, etc. H2-antihistamines include, for example, Cimetidine, Famotidine, Lafutidine, Nizatidine, Ranitidine, Roxatidine, Tiotidine, etc.

### Screening Candidate Agents

Screening methods include methods to optimize SCGB1D2 proteins and peptides derived therefrom, and to develop mimetics of SCGB1D2. Candidate agents of interest as mimetics are biologically active agents that encompass numerous chemical classes, primarily organic molecules, which may include organometallic molecules, inorganic molecules, genetic sequences, etc. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, N.Y., (1996), Ninth edition.

Samples of interest include compounds being assessed for potential therapeutic value, i.e. drug candidates. The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1. mu·I to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides, or targeted modifications of a SCGB1D2 protein or peptide derived therefrom. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for biological activity by adding the agent to at least one and usually a plurality of Borrelia species, and may be compared to SCGB1D2 protein activity. The change in readout in response to the agent is measured, desirably normalized, and may include readouts in the presence and absence of the factors, obtained with other agents, which may or may not include known inhibitors of known pathways, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Activity can be measured, for example, as shown in the Examples, and in vitro efficacy may be followed by a determination of activity in an animal model or in clinical trials.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Secretoglobin Family 1D Member 2 (SCGB1D2) Protein Inhibits Growth of Borrelia burqdorferi

Lyme disease (i.e., borreliosis) is an infectious disease caused by bacteria of the genus Borrelia, and transmitted by ticks. While most individuals have subsiding or short-term infection, others develop severe infection which requires intensive antibiotic treatment and may result in chronic illness. Seasonality of the disease is well characterized in Finland, and an increasing number of patients with Lyme disease have emerged during recent decades. Yet the biological risk factors and disease mechanisms for infection or severe illness are still only partially understood. Therefore, we performed a genome-wide association study (GWAS) on Lyme disease assessed by International Classification of Diseases (ICD)-9 and 10 codes and explored the phenotypic and genetic risk factors with the goal to finemap the most significant genetic associations and to understand underlying biology.

We utilized data from 342,499 individuals who have participated in the FinnGen project to estimate the effect of genetic variation for Lyme disease. Descriptive analysis showed that 44.3% of all participants were male in FinnGen. 5,248 (1.5%) of FinnGen participants had received a Lyme disease diagnosis between 1988 and 2021. 1,974 (37.6%) of the cases were male. The diagnoses were derived from ICD-codes in the Finnish national hospital and primary care registries (Table 1).

### Results

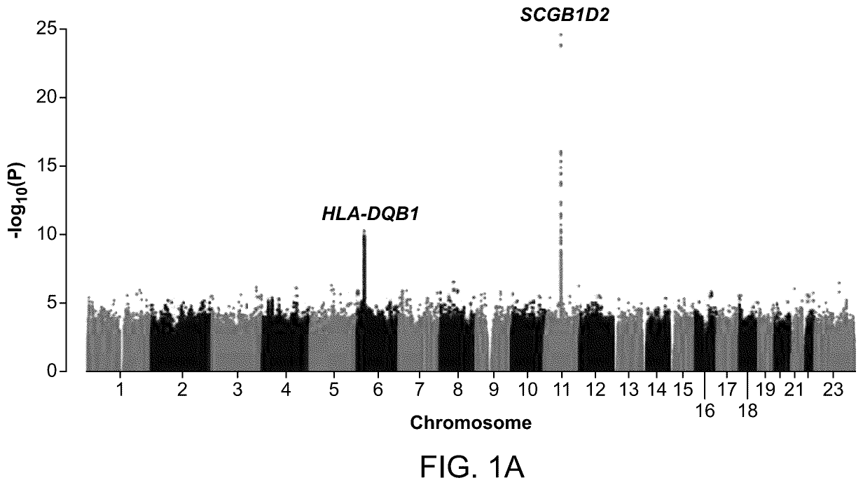

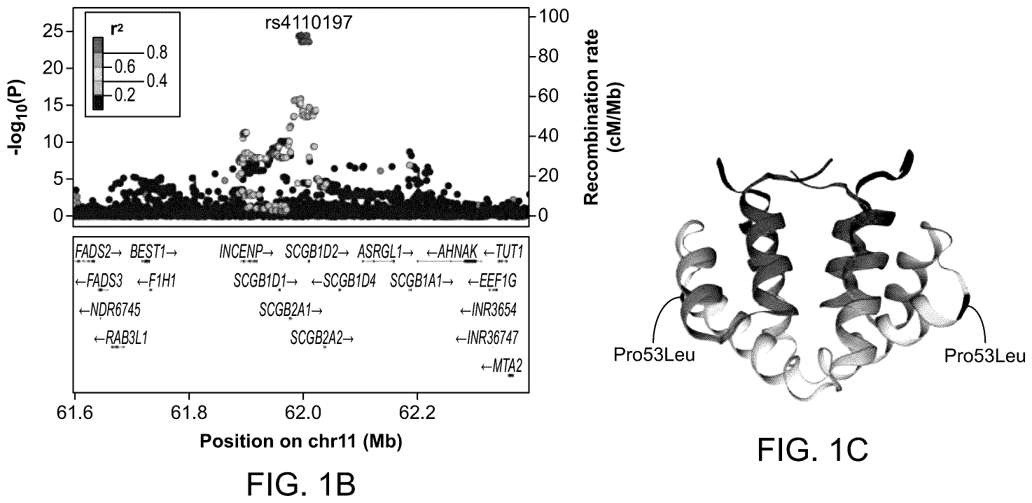

Human leukocyte antigen loci affect susceptibility to Lyme disease. We identified two genetic loci in a GWAS for Lyme disease (P<5.0×10⁻⁸, FIG. 1 and Table 2). One of these was located at the human leukocyte antigen (HLA) region on chromosome 6. HLA can affect predisposition to infectious and autoimmune traits, and this association highlights the overall importance of the HLA in Lyme disease. Furthermore, the lead variant (rs9273375, P=6.53×10 ⁻¹¹) is located at the 3′ end of HLA-DQB1 gene. In addition, finemapping of this association shows that the lead variant, rs9273375, likely reflects the signal from HLA-DQB1*06:02 (dominant model's P=3.1×10⁻¹⁰, r²=0.888 between rs9273375 and DQB1*06:02, Table 3). The specific alleles, and especially the HLA-DQB1*06:02 allele or alleles in high linkage disequilibrium (LD) such as HLA-DRB1*15:01, have been previously associated with influenza-A infection, autoimmune diseases such as multiple sclerosis, and with type-1 narcolepsy.

Missense variants at SCGB1D2 affect susceptibility to Lyme disease. While HLA association provides a proof of principle establishing Lyme disease as an infectious trait that is modified by genetic risk factors, the strongest and most compelling association was found at Secretoglobin family 1D member 2 locus (SCGB1D2, rs4110197, P=2.95×10⁻²⁵). rs4110197 is an intronic variant in SCGB1D2 and finemapping of the locus indicated the causal variant is most likely among a set of eight single nucleotide polymorphisms (SNPs) including a common missense variant, rs2232950 (P=1.82×10⁻²⁴) with alternative allele frequency 40%, that is in high LD with the lead variant (r²=0.95). rs2232950 at position 158C>T of SCGB1D2 results in a substitution from proline to leucine (Pro53Leu) and predicted deleterious by SIFT and Polyphen algorithms.

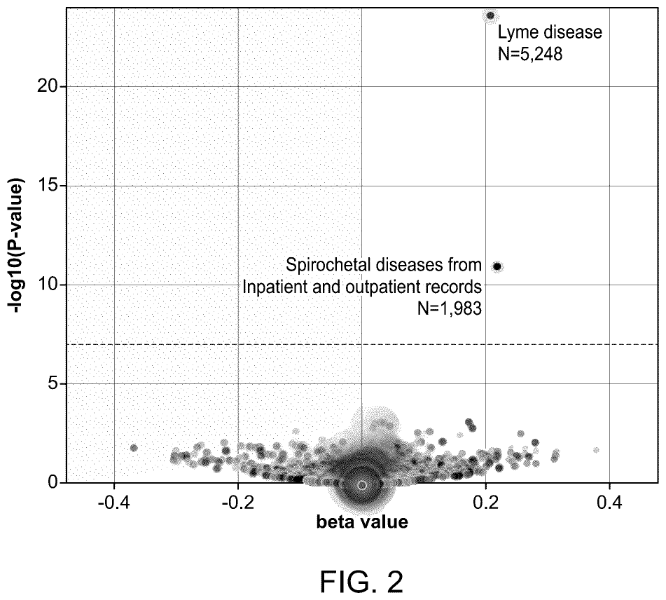

To understand the possible function of the variant, we examined its association in phenome-wide analysis (PheWAS) across 2,202 disease endpoints in FinnGen and publicly available GWAS data (FIGS. 1-2). In addition to Lyme disease, we observed association with hospitalized spirochetal infections (FIG. 2). As the spirochetal diseases include Lyme disease, we examined if the association was due to hospitalized Lyme disease patients and observed that the majority of hospitalized individuals had Lyme disease (N=1,765 of 1,983 individuals with hospital level spirochetal infection).

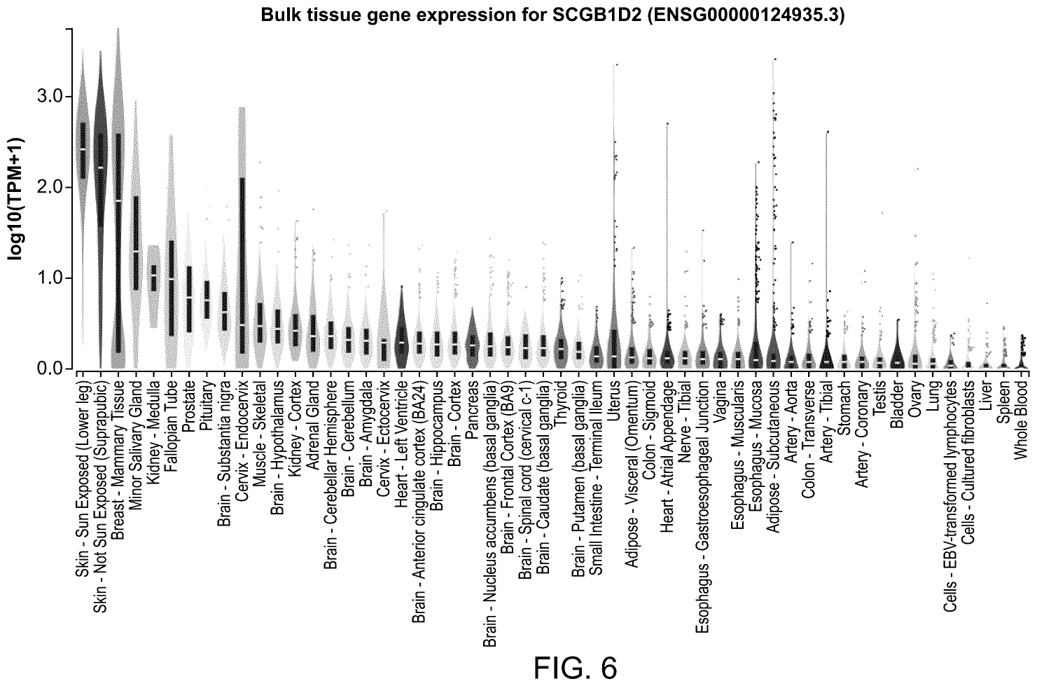

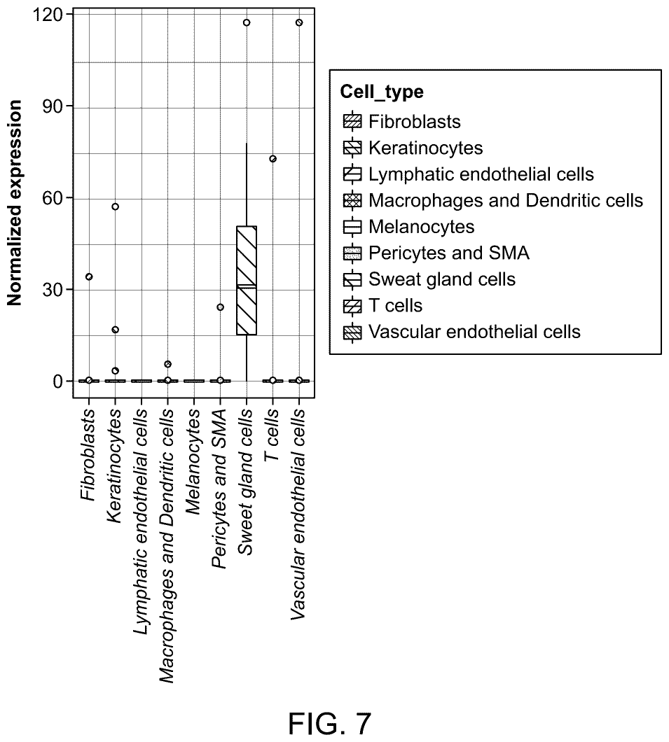

SCGB1D2 is expressed in the skin and secreted by sweat gland cells. SCGB1D2 belongs to a Secretoglobin protein family in which all members, except for SCGB1D2, are found in upper airway tissues and are generally expressed by secretory tissues of barrier organs. We used data from The Genotype-Tissue Expression (GTEx) release 8 to understand the tissue distribution of SCGB1D2. GTEx contains RNA expression samples from 948 donors across 54 tissues. The highest expression of SCGB1D2 occurred in two skin tissues, sun exposed and not sun exposed skin (transcripts per million (TPM)=258.2 and TPM=163.2, respectively, FIG. 6). Expression was also observed in other secretory tissues including mammary tissue and the uterus. As skin is the first tissue for Bb exposure, we were interested in understanding the cell type distribution of SCGB1D2 expression in the skin. Indeed, earlier studies have suggested that SCGB1D2 expression may be even specific to sweat glands in the skin. Therefore, we visualized previously published single cell sequencing data across cell types that are observed in the skin: fibroblasts, keratinocytes, lymphatic endothelial cells, melanocytes, pericytes and smooth muscle cells, sweat gland cells, T cells and vascular endothelial cells. The visualization showed that SCGB1D2 expression was specific to the sweat gland cells (FIG. 7) suggesting that SCGB1D2 may be secreted on the skin as part of sweat. Antimicrobial peptides such as dermcidin, lysozyme, lactoferrin, psoriasin, cathelicidin and β-defensins have been previously discovered in human sweat, and raises the possibility that SCGB1D2 may have antimicrobial properties.

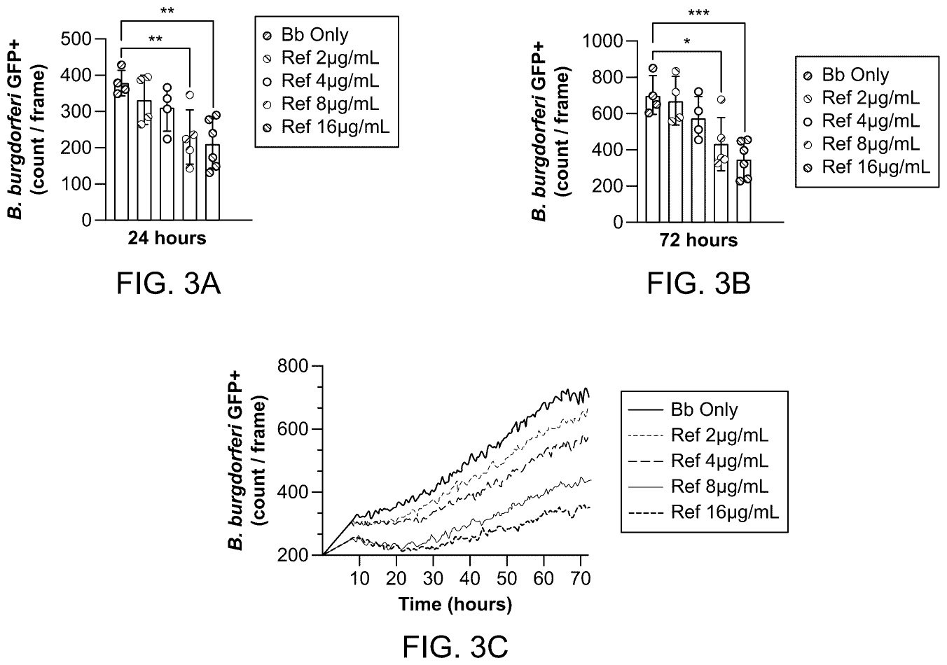

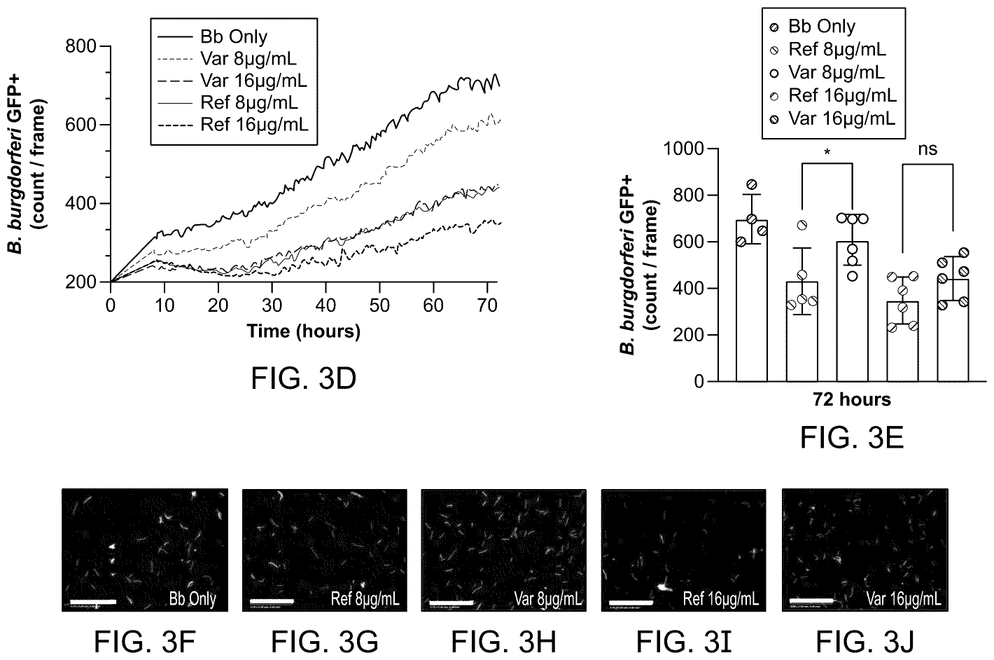

SCGB1D2 inhibits growth of B. burgdorferi. To investigate the impact of SCGB1D2 protein encoded by the reference genotype on Bb growth we performed a Bb growth assay using recombinant reference SCGB1D2. We discovered that SCGB1D2 inhibited the growth of Bb at 24 h (ANOVA F (4, 18)=5.77, P=0.0036) with significant growth inhibition at 8 μg/mL and 16 μg/mL concentrations (P=0.0091, P=0.0025 respectively, FIG. 3a). The growth inhibition was also significant at 72 h (ANOVA F (4, 18)=7.67, P=0.0009) time point at 8 μg/mL and 16 μg/mL concentrations (P=0.0137, P=0.0010, respectively, FIG. 3b).

Furthermore, despite an increase in the Bb count over time the growth inhibition was still notable at 72 hours suggesting that the effect is not transient (FIG. 3a-c). Furthermore, the growth inhibition was dose dependent at various SCGB1D2 concentrations over time (FIG. 3c).

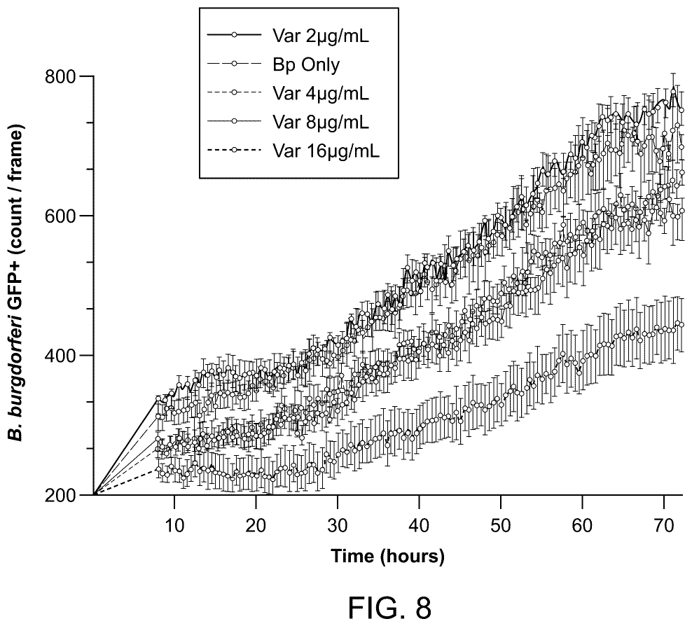

We then tested whether the SCGB1D2 P53L amino acid substitution encoded by the variant genotype might affect the SCGB1D2 protein function or even have a different impact on Bb growth compared to the reference SCGB1D2 protein without P53L substitution. To test this, we performed a Bb growth assay using recombinant reference SCGB1D2 compared to recombinant variant SCGB1D2 P53L. We discovered that approximately twice the amount of the variant SCGB1D2 P53L was needed to achieve similar inhibition to SCGB1D2 and that the variant SCGB1D2 P53L inhibited Bb growth only at the highest concentration of 16 μg/mL (FIG. 3d, FIG. 8). Similarly, while the reference SCGB1D2 overall inhibited Bb growth to a greater extent than SCGB1D2 P53L variant at 8 mg/mL, (P=0.046, FIG. 3) both reference SCGB1D2 and SCGB1D2 P53L variant were able to inhibit Bb growth at the highest concentration where the effect was possibly saturated (P=0.119, FIG. 3d-e).

We observed that in the presence of 16 μg/mL of variant SCGB1D2 P53L, and 8 μg/mL or 16 μg/mL of reference SCGB1D2, the Bb count reduces slightly from the starting amount to a trough around the 24-hour time point (FIG. 3d). This may be indicative of bacterial killing by SCGB1D2, especially since this graphical trough is not observed in the Bb only condition or with 8 μg/mL of variant SCGB1D2 P53L.

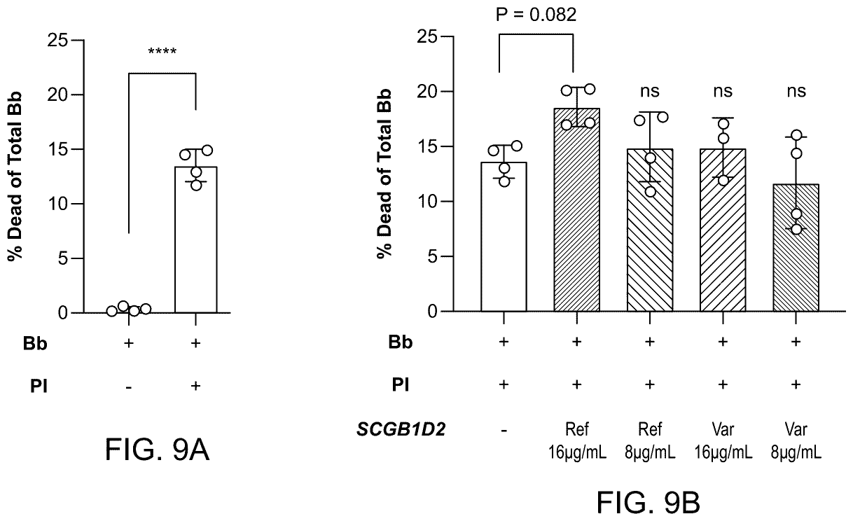

In order to label dead cells and differentiate bacterial death from growth inhibition, propidium iodide (PI) was included in a repeat experiment of Bb growth inhibition. We observed a presence of dead spirochetes in the assay, and the cytotoxicity of prolonged PI exposure. Furthermore, a significant proportion of Bb were bound to PI compared to Bb conditions without PI, even in the absence of SCGB1D2 proteins (FIG. 9a). To account for the increased PI binding, we examined the proportion of PI bound to Bb in the presence of either SCGB1D2 reference or variant protein but did not observe significant difference of Bb death at 24 hours compared to Bb with PI control (FIG. 9b). Nevertheless, the graphical trends of Bb death in the presence of SCGB1D2 protein were similar to observations of growth inhibition (FIG. 3).

Specifically, 16 μg/mL of reference SCGB1D2, which resulted in the most growth inhibition, also resulted in most PI binding (P=0.0822). Further investigation is required to determine SCGB1D2 mechanism of action against Bb and to differentiate bacterial killing from growth inhibition. Representative images of Bb from each treatment condition after 140 hours are also shown (FIG. 3f-3j).

In this study, we report the first genetic variants that affect Lyme disease susceptibility. Most notably, we identify a novel association with a deleterious missense variant at the SCGB1D2 gene. It is noteworthy that this SCGB1D2 P53Lvariant appears quite specific for Lyme disease and has not been previously reported as associated with any other disease, phenotype, or infection. Using expression and single cell analysis, we observe that SCBG1D2 has the highest expression in the skin and sweat gland cells. Furthermore, we characterized the function of SCGB1D2 protein and show that recombinant SCGB1D2 significantly inhibits Bb growth in vitro, and that around twice as much SCGB1D2 variant protein is required to achieve the same level of Bb growth inhibition as reference SCGB1D2. These findings demonstrate that SCGB1D2 is a restriction factor for Bb growth, and that SCGB1D2 variant has a reduced antibacterial function against Bb. Overall, our results elucidate novel biology and indicate a mechanism by which a secretoglobin provides protection against Bb infection in the skin. In the scope of infectious diseases, such disease-specific associations help teach us about specific host defenses that impact Lyme disease.

The current standard of care treatment for Lyme disease is bacteriostatic antibiotics such as amoxicillin or doxycycline, which restrict growth of bacteria similarly to SCGB1D2. Therefore, further investigating the prophylactic potential of SCGB1D2 to prevent Lyme disease by restricting Bb growth and dissemination in early disease stages, is of high interest, especially in individuals carrying the P53L mutation in SCGB1D2. SCGB1D2 can be applied topically as an intervention to reduce risk of Lyme disease. Similarly, SCGB1D2 and similar molecules may open novel avenues for treating late-stage Lyme disease.

Also in our study, we identified a novel association with Lyme disease and HLA-DQB1*06:02. This finding highlights the overall impact that HLA alleles have on infectious and autoimmune traits and raises two interesting points. First, the balance between innate and adaptive immune responses in clearing Bb infection in the human host remains an active topic of research. HLA class II alleles such as DQB1*06:02 are critical in modulating adaptive immune responses, and our findings suggest that adaptive immune responses, through primarily T-cell or B-cell mediated immunity, have an important role in Lyme disease. Second, both HLA-DRB1*15:01 and DQB1*06:02 have been previously implicated in brain autoimmune and infectious diseases such as multiple sclerosis, narcolepsy and Influenza-A. HLA-DRB1 has additionally been implicated in Lyme arthritis, and specifically the HLA-DRB1*15:01 allele was found to be more common in antibiotic-refractory Lyme arthritis patients than antibiotic-responsive patients. Our findings provide yet another infectious disease trait that is associated with these same HLA alleles.

Finally, our findings provide a compelling novel association with SCGB1D2 protein as a restriction factor secreted in the skin and sweat. Additionally, they indicate a novel host defense mechanism against Borrelia infection and against Lyme disease. This finding provides a novel therapeutic avenue for drug development to prevent and treat Lyme disease.

### Methods

Data in FinnGen. FinnGen is a joint research project of the public and private sectors, launched in Finland in the autumn of 2017, that aims to genotype 500,000 Finns including prospective and retrospective epidemiological and disease-based cohorts as well as hospital biobank samples. FinnGen combines genome data with longitudinal health care registries using unique personal identification codes allowing data collection and follow-up even over the whole life span. The FinnGen data release 8 is composed of 342,499 Finnish participants. The diagnosis of Lyme disease was based on ICD-codes (ICD-10: A69.2, ICD-9: 1048A), which were obtained from the Finnish national hospital and primary care registries including 5,248 individuals with Lyme disease and 337,251 controls. Main characteristics of the participants are outlined in Table 1.

Genotyping and imputation in FinnGen. FinnGen samples were genotyped with Illumina and Affymetrix chip arrays (Illumina Inc., San Diego, and Thermo Fisher Scientific, Santa Clara, CA, USA). Genotype calls were made with GenCall and zCall algorithms for Illumina and AxiomGT1 algorithm for Affymetrix data. Chip genotyping data produced with previous chip platforms and reference genome builds were lifted over to build version 38 (GRCh38/hg38).

In sample-wise quality control, individuals with ambiguous gender, high genotype missingness (>5%), excess heterozygosity (±4 standard deviation) and non-Finnish ancestry were excluded. In variant-wise quality control variants with high missingness (>2%), low Hardy-Weinberg equilibrium P-value (<1.0×10⁻⁶) and minor allele count<3 were excluded. Prior imputation, chip genotyped samples were pre-phased with Eagle 2.3.5 with the default parameters, except the number of conditioning haplotypes was set to 20,000.

Genotype imputation was done with the population-specific SISu v4 reference panel. Variant call set was produced with Genomic analyses toolkit (GATK) HaplotypeCaller algorithm by following GATK best-practices for variant calling. Genotype-, sample- and variant-wise quality control was applied in an iterative manner by using the Hail framework v0.1 and the resulting high-quality whole genome sequenced data for 3,775 individuals were phased with Eagle 2.3.5. Post-imputation quality control involved excluding variants with INFO score<0.7.

Statistical methods. To compare the main characteristics of the participants and finemap HLA locus, a multivariate logistic regression model was used. A genome-wide association study was performed by Scalable and Accurate Implementation of Generalized mixed model (SAIGE) with a saddle point approximation to calibrate unbalanced case-control ratios. Analysis was adjusted for current age or the age at death, sex, genotyping chip, genetic relationship and first 10 principal components. To examine SCGB1D2 locus and its genomic variation's causality to Lyme disease in more detail, we finemapped this region utilizing the “Sum of Single Effects”-model, called SuSiE. We performed a phenome-wide association analysis by retrieving association statistics for rs2232950 and all core phenotypes from FinnGen. We considered P-values significant if they passed Bonferroni correction for 2,202 tests corresponding to P-value 5.2×10⁻⁵. We examined RNA expression across tissue types using GTEx v8 using the fully processed and normalized gene expression matrixes for each tissue. These same values are used for eQTL calculations by GTEx.

We used previously published single cell sequencing data across cell types that are observed in the skin: fibroblasts, keratinocytes, lymphatic endothelial cells, melanocytes, pericytes and smooth muscle cells, sweat gland cells, T cells and vascular endothelial cells, and tested SCGB1D2 expression in these cell types.

Data for functional analysis of Bb was collected by the IncuCyte® S3 and analyzed in GraphPad Prism v9.2.0. We conducted one-way ANOVA to explore the differences between groups followed by Dunnett's post-hoc test to determine whether the tested groups were significantly different from the control group. We used independent T-tests in comparisons where there were only two groups.

B. burgdorferi growth inhibition assay. B31A3-GFP (Green Fluorescent Protein) Bb was cultured at 37° C. in Barbour-Stonner-Kelly with 4-(2-hydroxyethyl)-1-piperazineethanesLifonic acid (HEPES) buffer media (BSK-H) complete with 6% rabbit serum (Millipore Sigma) and 1% Amphotericin B (Sigma-Aldrich). Bacterial concentration was determined by flow cytometry (Becton Dickinson LSRFortessa) such that 150,000 spirochetes per well of an ImageLock 96-well plate (Sartorius) were incubated with either 8 μg/mL or 16 μg/mL of variant (SEQ ID NO:2 TP607036; MKLSVCLLLVTLALCCYQANAEFCPALVSELLDFFFISEPLFKLSLAKFDAPLEAVAAKLGVKRCT DQMSLQKRSLIAEVLVKILKKCSV) or reference (SEQ ID NO:1 TP607035; MKLSVCLLLVTLALCCYQANAEFCPALVSELLDFFFISEPLFKLSLAKFDAPPEAVAAKLGVKRCT DQMSLQKRSLIAEVLVKILKKCSV) SCGBID2 recombinant protein (Origene Technologies) in a total well volume of 150 uL. Incubation at 37° C. and 5% CO2 occurred inside of an IncuCyte® S3 (Sartorius) to measure real-time fluorescent intensity and capture phase images over time. Images were acquired using a 20× objective at 300-ms exposure per field of view. In order to limit false positive background fluorescence, threshold values to determine GFP⁺ events were set such that only Bb in phase and overlapped with green fluorescence events were counted.

A repeat Bb growth inhibition assay was performed as previously described, with the addition of 1.5 μL propidium iodide (Millipore Sigma) per well prior to incubation. After 24 hours of incubation, samples were fixed in 4% paraformaldehyde, resuspended in flow cytometry buffer (2% FBS, 1 mmol EDTA, in PBS), and analyzed by flow cytometry.

Demographic characteristics. For our study, we used the FinnGen cohort, which combines genomic data with national health registries.

We defined Lyme disease by extracting International Classification of Diseases (ICD)-9 (1048A) and ICD-10 (A69.2) codes from hospital inpatient, hospital outpatient and primary outpatient health registries. Our data consisted of 5,248 individuals with Lyme disease and 337,251 controls. Of all patients with Lyme disease diagnosis 37.6% were male and the mean age was 65.1 years. The mean age at the first Lyme disease diagnosis was 60.5 years (Table 1).

TABLE 1
Demographic and diagnosis information in individuals
diagnosed with Lyme disease in FinnGen

| | | | | | | P Lyme vs. |
|---|---|---|---|---|---|---|
| | Lyme | | Non-Lyme | | OR [95% CI] | Non-Lyme |
| N ICD-10 A69.2 | 5,189 | | | | | |
| N ICD-9 1048A | 59 | | | | | |
| N total | 5,248 | (1.5%) | 337,251 | | | |
| Sex (male) | 1,974 | (37.6%) | 149,646 | (44.4%) | 1.50 [1.41-1.58] | <2 × 10⁻¹⁶ |
| Age (mean, SD) | 65.1 | (14.3) | 59.0 | (18.0) | 1.02 [1.021-1.024] | <2 × 10⁻¹⁶ |

| | | |
|---|---|---|
| Age at first diagnosis | 60.5 | (15.1) |

for Lyme (mean, SD)

Comparison between individuals with and without Lyme disease diagnosis in FinnGen.
ICD = International Classification of Diseases, OR = odds ratio, CI = confidence interval, SD = standard deviation.

Analysis of individual genetic variants. To study genetics behind Lyme disease we analyzed a total of 342,499 samples from the FinnGen Data Freeze 8 with 5,248 individuals with Lyme disease diagnosis. For the GWAS we utilized Scalable and accurate implementation of generalized mixed model (SAIGE), which uses saddle point approximation to calibrate unbalanced case-control ratio. In addition, this method reduces the risk for type 1 error. Our GWAS analysis was adjusted for current age or the age at death, sex, genotyping chip, genetic relationship and first the 10 principal components.

The GWAS revealed two genome-wide significant signals (P<5.0×10⁻⁸). The characteristics of these loci are presented in Table 2. Our analysis pointed to a genome-wide signal in the HLA-region (rs9273375) located at the 3′ end of HLA-DQB1. In addition, we observed the strongest association in SCGB1D2 where the lead intronic variant (rs4110197) was found to be in high LD with a missense variant (rs2232950). Curiously, this variant causes amino acid change from proline to leucine, and this change is predicted deleterious by several databases.

TABLE 2
Genome-wide lead variants for Lyme disease

| | | | | | AF | Finnish | |
|---|---|---|---|---|---|---|---|
| CHR | rsid | REF | ALT | AF cases | controls | enrichment | OR [95% CI] |
| 6 | rs9273375 | G | C | 0.83 | 0.85 | 1.01 | 0.83 [0.79-0.88] |
| 11 | rs4110197 | C | T | 0.45 | 0.40 | 1.27 | 1.23 [1.19-1.29] |

Characterization of two genome-wide significant Lyme disease loci. Effect sizes and allele frequencies are reported in terms of alternative allele (ALT). Finnish enrichment is computed using the Genome Aggregation Database (gnomAD) data comparing Finnish individuals to other European populations. CHR = chromosome, REF = reference allele, AF = allele frequency, OR = odds ratio, CI = confidence interval.

### Understanding of Genetic Associations

HLA locus. HLA has a strong and established role in human immune defense. However, its contribution to Lyme disease has not been previously thoroughly explored. Our GWAS results showed a genome-wide significant HLA-locus, and to study this finding in more detail we finemapped this region. We computed association statistics with each HLA-allele from the HLA-A, HLA-B, HLA-C, HLA-DRB1, HLA-DQA1, HLA-DQB1, HLA-DPA1 and HLA-DPB1 genes and discovered the most significant association with HLA-DQB1*06:02. Similarly, our lead variant rs9273375 was in high linkage disequilibrium (LD) with HLA-DQB1*06:02 (r²=0.888). As HLA-DQB1*06:02 is also in high LD with DRB1*15:01, we estimated the pairwise LD also for HLA-DRB1*15:01. The analysis supported HLA-DQB1*06:02 as the most significant HLA-allele to associate with Lyme disease.

TABLE 3
Linkage disequilibrium between lead SNP at the HLA- alleles.

| Variant 1 | Variant 2 | r², D′ |
|---|---|---|
| rs9273375 | HLA-DQB1*06:02 | 0.888, 0.990 |
| rs9273375 | HLA-DRB1*15:01 | 0.877, 0.979 |
| HLA-DRB1*15:01 | HLA-DQB1*06:02 | 0.983, 0.997 |

Variant 1 and Variant 2 represent single variants and HLA alleles for LD estimate. We represent LD as r² and D′ for each comparison using the genotype data and imputed HLA alleles from FinnGen.

SCGB1D2 locus. Our GWAS result showed a novel genome-wide significant finding related to Lyme disease in chromosome 11. We found that the lead intronic variant (rs4110197) in SCGB1D2 was in high LD with a missense variant (rs2232950). This causes amino acid change from proline to leucine indicating a deleterious mutation by several algorithms. To examine SCGB1D2 locus and its genomic variation's causality to Lyme disease in more detail, we finemapped this region utilizing the “Sum of Single Effects”-model, called SuSiE. The 99% credible set included eight variants including the common missense variant rs2232950. Furthermore, SuSiE predicted posterior probability of 0.22 to the lead variant rs4110197.

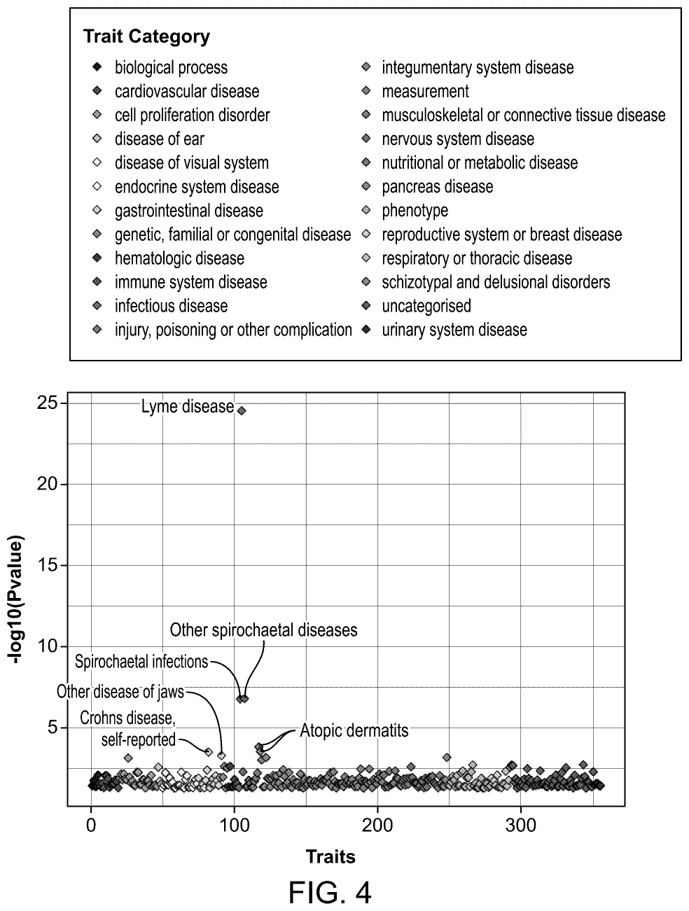

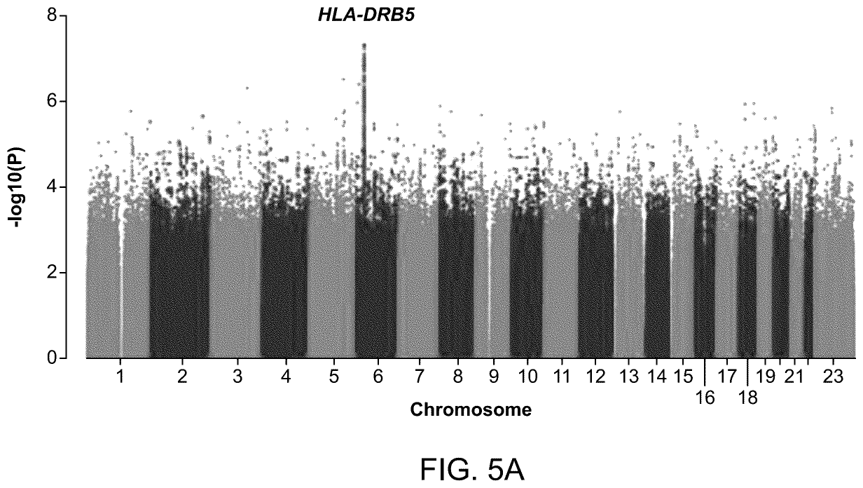

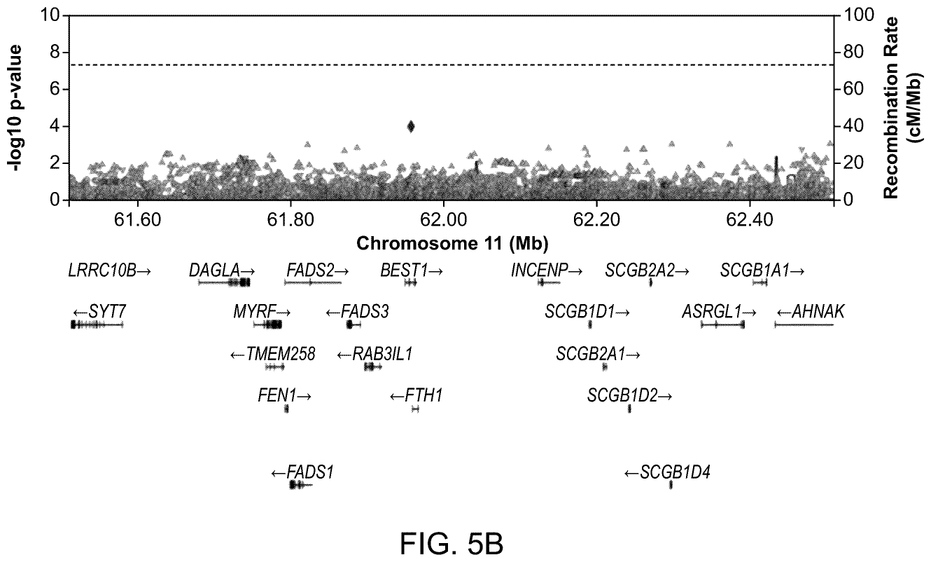

PheWAS analysis. Comparison with publicly available traits. We performed a phenome-wide association analysis (PheWAS) to explore the association between the missense variant rs2232950 and 2,202 disease endpoints from FinnGen. FinnGen endpoints include primarily electronic health record derived phenotypes. To complement this analysis, we computed PheWAS also using the OpenTargets platform, which includes traits from publicly available GWASes and traits from other biobanks. This analysis did not reveal additional significant associations besides its association with Lyme disease in FinnGen and rs2232950. Therefore, this analysis did not provide additional insight into the function of SCGB1D2 (FIG. 4).

GWAS for syphilis. We were curious to investigate whether we could find associations between our findings in SCGB1D2 and other fairly common spirochetal infections, such as syphilis. Therefore, we performed a GWAS for syphilis using FinnGen data with 719 individuals with syphilis ICD-codes captured from hospital inpatient, outpatient and primary care registries (FIG. 4). We observed a significant association with the HLA-DRB5 locus but did not find a significant association between SCGB1D2 locus and syphilis at single variant or locus level (rs2232950, P=0.6). This finding further supported that rs2232950 may be a specific predisposing component to Lyme disease.

GTEx. We examined RNA expression across tissue types using The Genotype-Tissue Expression (GTEx) v8 using the fully processed and normalized gene expression matrices for each tissue. These data contain RNA expression samples from 948 donors across 54 tissues. These same values are used for eQTL calculations by GTEx. We extracted the values for SCGB1D2 in these data and plotted the normalized values per tissue (FIG. 6). The two skin types (sun exposed and unexposed) show the highest expression levels of SCBG1D2.

In order to understand the relevant cell types for SCGB1D2 expression from the skin we examined single cell sequencing data from skin. We observed that SCGB1D2 was predominantly expressed by the sweat gland cells. The results support our main findings in live Bb. We estimate the effect of SCGB1D2 P53L on Bb growth and examine the killing capability of SCGB1D2 recombinant protein on live Bb.
Steere, A. C., Schoen, R. T. & Taylor, E. The clinical evolution of Lyme arthritis. Ann Intern Med 107, 725-731, doi:10.7326/0003-4819-107-5-725 (1987).
Wormser, G. P. et al. The clinical assessment, treatment, and prevention of lyme disease, human granulocytic anaplasmosis, and babesiosis: clinical practice guidelines by the Infectious Diseases Society of America. Clin Infect Dis 43, 1089-1134, doi:10.1086/508667 (2006).
Sajanti, E. et al. Lyme Borreliosis in Finland, 1995-2014. Emerg Infect Dis 23, 1282-1288, doi:10.3201/eid2308.161273 (2017).
Rizzoli, A. et al. Lyme borreliosis in Europe. Euro Surveil 16 (2011).
Tian, C. et al. Genome-wide association and HLA region fine-mapping studies identify susceptibility loci for multiple common infections. Nat Commun 8, 599, doi:10.1038/s41467-017-00257-5 (2017).
Lenz, T. L. et al. Widespread non-additive and interaction effects within HLA loci modulate the risk of autoimmune diseases. Nat Genet 47, 1085-1090, doi:10.1038/ng.3379 (2015).
Hammer, C. et al. Amino Acid Variation in HLA Class II Proteins Is a Major Determinant of Humoral Response to Common Viruses. Am J Hum Genet 97, 738-743, doi:10.1016/j.ajhg.2015.09.008 (2015).
Narwaney, K. J. et al. Association of HLA class II genes with clinical hyporesponsiveness to trivalent inactivated influenza vaccine in children. Vaccine 31, 1123-1128, doi:10.1016/j.vaccine.2012.12.026 (2013).
International Multiple Sclerosis Genetics, C. et al. Genetic risk and a primary role for cell-mediated immune mechanisms in multiple sclerosis. Nature 476, 214-219, doi:10.1038/nature10251 (2011).
Mignot, E. et al. Complex HLA-DR and -DQ interactions confer risk of narcolepsy-cataplexy in three ethnic groups. Am J Hum Genet 68, 686-699, doi:10.1086/318799 (2001).
Wang G, S. A., Carbonetto P, Stephens M. A Simple New Approach to Variable Selection in Regression, with Application to Genetic Fine Mapping. Journal of the Royal Statistical Society: Series B (Statistical Methodology). 82, 1273-1300 (2020).
Ng, P. C. & Henikoff, S. SIFT: Predicting amino acid changes that affect protein function. Nucleic Acids Res 31, 3812-3814, doi:10.1093/nar/gkg509 (2003).
Adzhubei, I. A. et al. A method and server for predicting damaging missense mutations. Nat Methods 7, 248-249, doi:10.1038/nmeth0410-248 (2010).
Jackson, B. C. et al. Update of the human secretoglobin (SCGB) gene superfamily and an example of ‘evolutionary bloom’ of androgen-binding protein genes within the mouse Scgb gene superfamily. Hum Genomics 5, 691-702, doi:10.1186/1479-7364-5-6-691 (2011).
Lu, X. et al. The cytokine-driven regulation of secretoglobins in normal human upper airway and their expression, particularly that of uteroglobin-related protein 1, in chronic rhinosinusitis. Respir Res 12, 28, doi:10.1186/1465-9921-12-28 (2011).
Consortium, G. T. The Genotype-Tissue Expression (GTEx) project. Nat Genet 45, 580-585, doi:10.1038/ng.2653 (2013).
He, H. et al. Single-cell transcriptome analysis of human skin identifies novel fibroblast subpopulation and enrichment of immune subsets in atopic dermatitis. J Allergy Clin Immunol 145, 1615-1628, doi:10.1016/j.jaci.2020.01.042 (2020).
Na, C. H. et al. Integrated Transcriptomic and Proteomic Analysis of Human Eccrine Sweat Glands Identifies Missing and Novel Proteins. Mol Cell Proteomics 18, 1382-1395, doi:10.1074/mcp.RA118.001101 (2019).
Csosz, E., Emri, G., Kallo, G., Tsaprailis, G. & Tozser, J. Highly abundant defense proteins in human sweat as revealed by targeted proteomics and label-free quantification mass spectrometry. J Eur Acad Dermatol Venereol 29, 2024-2031, doi:10.1111/jdv.13221 (2015).
Lantos, P. M. et al. Clinical Practice Guidelines by the Infectious Diseases Society of America (IDSA), American Academy of Neurology (AAN), and American College of Rheumatology (ACR): 2020 Guidelines for the Prevention, Diagnosis and Treatment of Lyme Disease. Clin Infect Dis 72, 1-8, doi:10.1093/cid/ciab049 (2021).
Loh, P. R. et al. Reference-based phasing using the Haplotype Reference Consortium panel. Nat Genet 48, 1443-1448, doi:10.1038/ng.3679 (2016).
Zhou, W. et al. Efficiently controlling for case-control imbalance and sample relatedness in large-scale genetic association studies. Nat Genet 50, 1335-1341, doi:10.1038/s41588-018-0184-y (2018).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method for inhibiting the growth of Borrelia species, the method comprising:
contacting the Borrelli sp. with an effective dose of a human secretoglobin family 1D member 2 (SCGB1D2) agent.

2. The method of claim 1, wherein the Borrelia sp. is a cause of Lyme disease.

3. The method of claim 1, wherein the Borrelia sp. is B. burgdorferi sensu stricto.

4. The method of claim 1, wherein the Borrelia sp. is one or more of Borrelia afzelli, B. garinii, B. bavariensis, and B. spielmanii.

5. The method of claim 1, wherein the SCGB1D2 agent is a human reference sequence SCGB1D2 protein.

6. The method of claim 1, wherein the SCGBID2 agent is an agent that competes for binding to a human reference sequence SCGB1D2 protein.

7. The method of claim 1, wherein the SCGB1D2 agent is a mimetic of human SCGB1D2 protein.

8. The method of claim 1, wherein the SCGB1D2 agent is a biologically active fragment of human SCGB1D2 protein.

9. The method of claim 1, wherein the SCGB1D2 agent is a variant of human SCGB1D2 protein.

10. The method of claim 1, wherein the SCGB1D2 agent is administered systemically to an individual.

11. The method of claim 1, wherein the SCGB1D2 agent is administered topically to an individual.

12. The method of claim 1, wherein the SCGB1D2 agent is administered prophylactically to an individual.

13. The method of claim 1, wherein the SCGB1D2 agent is administered at a concentration of from about 0.1 mg/ml up to about 1000 mg/ml.

14. The method of claim 1, wherein the SCGB1D2 agent is administered to an individual at risk of a Borrelia infection.

15. The method of claim 1, wherein the SCGB1D2 agent is administered to an individual diagnosed with a Borrelia infection.

16. The method of claim 1, wherein the SCGB1D2 agent is administered to an individual that is heterozygous or homozygous for a variant allele of SCGB1D2 comprising a substitution of proline at position 53 to leucine (P53L).

17. A method of screening a candidate agent for efficacy in inhibition of growth of Borrelia species, the methods comprising designing an agent to mimic SCGB1D2 activity, and determining its effectiveness in inhibition of growth.

18. The method of claim 17, wherein the candidate agent is a mutagenized SCGB1D2 protein or peptide derived therefrom.
